(19) United States
(12) Patent Application Publication
(10) Pub. No.: US 2004/0019266 A1
(43) Pub. Date: Jan. 29, 2004

(54) Apparatus and method for radiopaque coating for an ultrasonic medical device

(75) Inventors: Rebecca I. Marciante, North Reading, MA (US); Anthony W. O'Leary, Walpole, MA (US); Bradley A. Hare, Chelmsford, MA (US); Mark J. Varady, Holliston, MA (US); Anita J. Chung, Cambridge, MA (US)

Correspondence Address: PALMER & DODGE, LLP, RICHARD B. SMITH, 111 HUNTINGTON AVENUE, BOSTON, MA, 02199, US

(73) Assignee: OmniSonics Medical Technologies, Inc.

(21) Appl. No.: 10/207,468

(22) Filed: Jul. 29, 2002

(51) Int. Cl. A61B 5/05

(52) U.S. Cl. 600/407

(57) ABSTRACT

The present invention provides an apparatus and a method for using an elongated ultrasonic probe in conjunction with a radiopaque coating in order to improve the visibility of the ultrasonic probe during a procedure such as fluoroscopy. The radiopaque coating may be an ink comprising an adhesive material. The adhesive material comprises a substance which allows for a significant amount of x-ray absorption. The present invention provides an ultrasonic device comprising an elongated probe having a small-diameter wherein the elongated probe is coated in a radiopaque coating. The present invention provides a method of improving the visibility of an ultrasonic device during a fluoroscopic procedure comprising applying a radiopaque coating to an elongated probe having a small diameter. The radiopaque coating of the present invention is capable of withstanding vibrations of the elongated probe and increases the visibility of the elongated probe in a fluoroscopic procedure.

### RELATED APPLICATIONS

None.

### FIELD OF THE INVENTION

The present invention relates generally to medical devices, and particularly to an apparatus and a method of radiopaque coatings for an ultrasonic medical device. The present invention relates to an ultrasonic medical device having an elongated probe with a biocompatible, non-toxic radiopaque coating that is capable of withstanding ultrasonic vibrations for the purpose of improving the visibility of the elongated probe during a fluoroscopic procedure, and a method for applying a radiopaque coating to an ultrasonic medical device.

### BACKGROUND OF THE INVENTION

Vascular occlusions (clots or thrombi and occlusional deposits, such as calcium, fatty deposits, or plaque) result in the restriction or blockage of blood flow in the vessels in which they occur. Occlusions result in oxygen deprivation (“ischemia”) of tissues supplied by these blood vessels. Prolonged ischemia results in permanent damage of tissues which can lead to myocardial infarction, stroke, or death. Targets for occlusion include coronary arteries, peripheral arteries and other blood vessels. The disruption of an occlusion or thrombolysis can be effected by pharmacological agents and/or mechanical means. However, many thrombolytic drugs are associated with side effects such as severe bleeding which can result in a cerebral hemorrhage. Mechanical methods of thrombolysis include balloon angioplasty, which can result in ruptures in a blood vessel, and is generally limited to larger blood vessels. Scarring of vessels is common, which may lead to the formation of a secondary occlusion (a process known as restenosis). Another common problem is secondary vasoconstriction (classic recoil), a process by which spasms or an abrupt closure of the vessel occurs. These problems are common in treatments employing interventional devices. In traditional angioplasty, for instance, a balloon catheter is inserted into the occlusion, and through the application of hydraulic forces in the range of ten to fourteen atmospheres of pressure, the balloon is inflated. The non-compressible balloon applies this significant force to compress and flatten the occlusion, thereby opening the vessel for blood flow. However, these extreme forces result in the application of extreme stresses to the vessel, potentially rupturing the vessel, or weakening it thereby increasing the chance of post-operative aneurysm, or creating vasoconstrictive or restenotic conditions. In addition, the particulate matter is not removed, rather it is just compressed. Other mechanical devices that drill through and attempt to remove an occlusion have also been used, and create the same danger of physical damage to blood vessels.

Ultrasonic probes are devices which use ultrasonic energy to fragment body tissue (see, e.g., U.S. Pat. No. 5,112,300; U.S. Pat. No. 5,180,363; U.S. Pat. No. 4,989,583; U.S. Pat. No. 4,931,047; U.S. Pat. No. 4,922,902; and U.S. Pat. No. 3,805,787) and have been used in many surgical procedures. The use of ultrasonic energy has been proposed both to mechanically disrupt clots, and to enhance the intravascular delivery of drugs to clot formations (see, e.g., U.S. Pat. No. 5,725,494; U.S. Pat. No. 5,728,062; and U.S. Pat. No. 5,735,811). Ultrasonic devices used for vascular treatments typically comprise an extracorporeal transducer coupled to a solid metal wire which is then threaded through the blood vessel and placed in contact with the occlusion (see, e.g., U.S. Pat. No. 5,269,297). In some cases, the transducer is delivered to the site of the clot, the transducer comprising a bendable plate (see, e.g., U.S. Pat. No. 5,931,805).

The ultrasonic energy produced by an elongated probe is in the form of very intense, high frequency sound vibrations that result in physical reactions in the water molecules within a body tissue or surrounding fluids in proximity to the probe. These reactions ultimately result in a process called “cavitation,” which can be thought of as a form of cold (i.e., non-thermal) boiling of the water in the body tissue, such that microscopic bubbles are rapidly created and destroyed in the water creating cavities in their wake. As surrounding water molecules rush in to fill the cavity created by collapsed bubbles, they collide with each other with great force. This process is called cavitation and results in shock waves running outward from the collapsed bubbles which can wear away or destroy material such as surrounding tissue in the vicinity of the elongated probe.

Some ultrasonic devices include a mechanism for irrigating an area where the ultrasonic treatment is being performed (e.g., a body cavity or lumen) in order to wash tissue debris from the area of treatment. Mechanisms used for irrigation or aspiration described in the art are generally structured such that they increase the overall cross-sectional profile of the elongated probe, by including inner and outer concentric lumens within the probe to provide irrigation and aspiration channels. In addition to making the probe more invasive, prior art probes also maintain a strict orientation of the aspiration and the irrigation mechanism, such that the inner and outer lumens for irrigation and aspiration remain in a fixed position relative to one another, which is generally closely adjacent to the area of treatment. Thus, the irrigation lumen does not extend beyond the suction lumen (i.e., there is no movement of the lumens relative to one another) and any aspiration is limited to picking up fluid and/or tissue remnants within the defined area between the two lumens.

Medical devices utilizing ultrasonic energy to destroy tissue in the human body are known in the art. A major drawback of existing ultrasonic devices comprising an elongated probe for tissue removal is that they are relatively slow in comparison to procedures that involve surgical excision. This is mainly attributed to the fact that such ultrasonic devices rely on imparting ultrasonic energy to contacting tissue by undergoing a longitudinal vibration of the probe tip, wherein the probe tip is mechanically vibrated at an ultrasonic frequency in a direction parallel to the probe longitudinal axis. This, in turn, produces a tissue destroying effect that is entirely localized at the probe tip, which substantially limits its ability to ablate large tissue areas in a short time.

One solution to the above-identified drawback is to vibrate the tip of the probe in a transverse direction—i.e. perpendicular to the longitudinal axis of the probe—in addition to vibrating the tip in the longitudinal direction. Such a device is capable of fragmenting and emulsifying tissue that has caused an occlusion within the interior of a blood vessel, and provides a method for removing such occlusions with high efficiency. Surprisingly, a similar result can be achieved by an ultrasonic device comprising a vibrating probe whose vibrations are restricted to occur exclusively in a transverse direction to the axis of the probe. By eliminating the axial motion of the probe and allowing transverse vibrations only, fragmentation of large areas of tissue spanning the entire length of the probe is possible due to the generation of multiple cavitational nodes along the probe's length, perpendicular to the axis of the probe. Such an ultrasonic device provides a rapid, highly efficient method for removing occlusions as compared with conventional devices and methods that have primarily utilized longitudinal vibration (along the axis of the probe) for tissue ablation.

An additional feature of an ultrasonic device operating in a transverse mode is the ability to employ probes of extremely small diameter as compared with previously disclosed devices without a loss of efficiency. Efficiency is maintained since the tissue fragmentation process is no longer dependent solely upon the area of the probe tip (the distal end). Highly flexible probes can therefore be obtained to mimic device shapes that enable facile insertion into highly occluded or extremely narrow interstices within a blood vessel.

The prior art has not solved the problem of a decrease in the visibility of the small-diameter, ultrasonic probe during a procedure deep in the body such as fluoroscopy, described below. Also, in order to achieve sufficient transverse vibrations along the length of the probe, the probe must be manufactured from a high capacitance material. Often, such high capacitance materials are non-radiopaque. Non-radiopaque materials allow the passage of x-rays or other radiation. Because these high capacitance materials do not absorb radiation, a user is unable to locate the exact position of the ultrasonic probe inside the human body during a fluoroscopic procedure.

Fluoroscopy is a study of moving body structures. A continuous x-ray beam is passed through the body part being examined, and is transmitted to a TV-like monitor so that the body part and its motion can be seen in detail. Fluoroscopy is used in many types of examinations and procedures, such as barium x-rays, cardiac catheterization, and placement of intravenous (IV) catheters (hollow tubes into veins or arteries). In barium x-rays, fluoroscopy allows the physician to see the movement of the intestines as the barium moves through them. In cardiac catheterization, fluoroscopy enables the physician to see the flow of blood through the coronary arteries in order to evaluate the presence of arterial blockages. For intravenous catheter insertion, fluoroscopy assists the physician in guiding the catheter into a specific location inside the body. Fluoroscopy helps diagnose problems with the digestive tract, the bowel, kidneys, gallbladder, stomach, upper GI and joints. Fluoroscopy is used during many diagnostic and therapeutic radiologic procedures, to observe the action of instruments being used either to diagnose or to treat the patient.

Fluoroscopic imaging is useful when it is necessary to radiograph a dynamic situation. Fluoroscopy is most commonly used to evaluate the gastrointestinal tract but can also be used to record the motion of any other body part in which the component in motion might be helpful in arriving at a diagnostic decision. A fluoroscope is a radiographic machine which has an x-ray tube mounted in a way that the beam can pass through the patient and be recorded on a fluorescent screen. In modern fluoroscopes, the observer does not look directly at the fluoroscope screen but looks at a video image produced from a video camera which is focused on the screen. These machines also incorporate a spot film device which will allow the operator to move a film into the beam and take “snap shot” pictures of any abnormality which is observed. This equipment is usually attached to an x-ray table which allows the operator to tilt the patient or camera in various directions and the x-ray tube is most commonly positioned under the table top with the spot film device and the fluorescent screen including an image intensifier being above the patient if the patient is lying supine on the table.

The prior art discloses past attempts to better visualize non-radiopaque materials once they have entered a human body during a medical procedure. U.S. Pat. No. 5,824,042 to Lombardi et al. discloses an endoluminal prosthesis for deployment in a body lumen of a patient's body, the prosthesis comprising a tubular fabric liner and a radially expandable frame supporting the liner. A plurality of imagable bodies are attached to the liner, the imagable bodies providing a sharp contrast so as to define a pattern which indicates the prosthesis position when the prosthesis is imaged within the patient body. Lombardi et al. requires the plurality of imagable bodies to be stitched into tubular fabric liner; the plurality of imagable bodies could not be stitched into an ultrasonic probe. The plurality of imagable bodies disclosed in Lombardi et al. would not be able to withstand vibrations of an ultrasonic device. Therefore, a need remains in the art for an apparatus and method of visualizing the position of an non-radiopaque, elongated probe during a procedure such as fluoroscopy.

U.S. Pat. No. 5,622,170 to Schulz discloses a system for sensing at least two points on an object for determining the position and orientation of the object relative to another object. Two light emitters mounted in spaced relation to each other on an external portion of an invasive probe, remaining outside an object into which an invasive tip is inserted, are sequentially strobed to emit light. In Schulz, a computer determines the position and orientation of the invasive portion of the probe inside the object by correlating the position of the invasive portion of the probe relative to a predetermined coordinate system with a model of the object defined relative to the predetermined coordinate system. Schulz does not allow for the position of the non-radiopaque, elongated probe to be determined directly but rather provides a representation of the probe's position relative to a predetermined coordinate system. Also, Schulz discloses an expensive, complicated and complex method of approximating the position of a probe once inside a body. Therefore, a need remains in the art for an apparatus and method of visualizing the position of an ultrasonic probe during a procedure such as fluoroscopy.

U.S. Pat. No. 5,588,432 to Crowley discloses an acoustic imaging system for use within a heart comprising a catheter, an ultrasound device incorporated into the catheter, and an electrode mounted on the catheter. In Crowley, a central processing unit creates a graphical representation of the internal structure, and superimposes items of data onto the graphical representation at locations that represent the respective plurality of locations within the internal structure corresponding to the plurality of items of data. Like Schulz, Crowley does not allow for the position of the medical device to be determined directly, but rather provides a representation of the device's position corresponding to the plurality of items of data. Therefore, a need remains in the art for an apparatus and a method of visualizing the position of a non-radiopaque, elongated probe during a procedure such as fluoroscopy.

Other attempts to improve the visibility of non-radiopaque devices include attaching a number of metal bands or the use of the non-radiopaque device in conjunction with a barium-filled catheter. Although such devices may improve the visibility of a non-radiopaque material, they are difficult to use in conjunction with a non-radiopaque ultrasonic probe because the metal bands are difficult to attach to an ultrasonic probe. A barium-filled catheter allows for improved visibility of the catheter, but does not allow for the exact location of the ultrasonic probe to be determined. Also, barium-filled catheters are known in the art to obstruct the visibility of surrounding arteries and veins. Therefore, a need remains in the art for an apparatus and a method of better visualizing the position of a non-radiopaque, elongated probe during a procedure such as fluoroscopy for improved safety and efficiency of the medical procedure.

Other attempts at improving the visibility of a non-radiopaque material include using a high-vacuum deposition process that results in a thin-film coating. Traditional ion-beam-assisted deposition (IBAD) employs an electron-beam evaporator to create a vapor of atoms that coats the surface of the device. A similar process known as microfusion comprises placing the substrate to be coated between two magnetrons. Provision is made for an adjustable bias to be applied to the substrate, as required, to control ion energy and flux. The prior art processes are complex, difficult to implement, and expensive. Therefore, a need remains in the art for a simple and inexpensive apparatus and a method of visualizing the position of a non-radiopaque, elongated probe during a procedure such as fluoroscopy.

The prior art devices and methods of visualizing a non-radiopaque, elongated probe once inside a body are complex, complicated and expensive. Therefore, there is a need in the art for developments in the visualization of non-radiopaque probes after entering the body. In particular, an apparatus and a method of treating a non-radiopaque ultrasonic probe so that the elongated probe does not lose the ability to oscillate in a transverse mode, but may gain the ability to be visualized during a medical procedure, such as fluoroscopy, would further advance the state of the art.

### SUMMARY OF THE INVENTION

The present invention provides an apparatus and a method for using an ultrasonic medical device comprising a non-radiopaque, elongated probe in conjunction with a biocompatible, non-toxic radiopaque coating in order to improve the visibility of the elongated probe during a procedure such as fluoroscopy. The radiopaque coating of the present invention may be an ink having an adhesive material. The adhesive material includes a substance which allows for a significant amount of x-ray absorption.

The present invention provides an ultrasonic device comprising a non-radiopaque, elongated probe having a small-diameter wherein the elongated probe is coated in a radiopaque coating. The radiopaque coating of the present invention is capable of withstanding ultrasonic vibrations of the elongated probe and the radiopaque coating increases the visibility of the probe in a procedure such as fluoroscopy. The non-radiopaque, elongated probe coated with a radiopaque coating allows the ultrasonic device to continue to benefit from the high capacitance properties of a non-radiopaque probe and gain the ability to absorb radiation and therefore increase the visibility of the elongated probe during a procedure such as fluoroscopy.

The present invention provides a method of improving the radiopacity of an ultrasonic device comprising applying a radiopaque coating to a non-radiopaque, elongated, ultrasonic probe having a small-diameter and viewing the elongated probe during a fluoroscopic procedure. The radiopaque coating of the present invention is capable of withstanding vibrations of the ultrasonic probe and increases the visibility of the probe in the fluoroscopic procedure.

The present invention is an apparatus comprising a non-radiopaque ultrasonic probe coated with a radiopaque coating. Utilizing a radiopaque coating with a non-radiopaque ultrasonic probe allows the apparatus to benefit from the high capacitance of the non-radiopaque material which will facilitate a series of transverse vibrations in the ultrasonic probe while allowing the probe to be visualized in a fluoroscopic procedure.

The ultrasonic probe of the present invention may be coated with a radiopaque coating at a plurality of predetermined locations on the elongated probe. Applying the radiopaque coating to a plurality of predetermined locations along the ultrasonic probe allows the user to visualize the plurality of predetermined locations along the probe while the probe is inserted in the body. Visualizing the plurality of predetermined locations along the ultrasonic probe allows the user to better control the location of the probe. Allowing the user to better visualize the location of the probe inside the body leads to increased safety and more efficient procedures.

The elongated probe of the present invention may comprise an amount of radiopaque ink suitable for a one time use of the ultrasonic device. Alternatively, the elongated probe may comprise an amount of radiopaque ink wherein the amount of ink allows the probe to be used a plurality of times.

### DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1A is a side plan view of an ultrasonic medical device of the present invention capable of operating in a transverse mode.

FIG. 1B is a side plan view of an ultrasonic medical device operating in a transverse mode of the present invention showing a plurality of nodes and a plurality of anti-nodes along an active area of an elongated probe.

FIG. 2 is a fragmentary view of an active end of an elongated probe coated with a radiopaque coating at a plurality of predetermined locations of the present invention.

FIG. 3 is an enlarged, fragmentary view of an elongated probe of FIG. 2 showing a distal end of the elongated probe having a small diameter.

FIG. 4 is a fragmentary view of an alternative embodiment of an elongated probe coated with a radiopaque coating at a plurality of predetermined locations of the present invention.

FIG. 5 is an enlarged, fragmentary view of an alternative embodiment of an elongated probe of FIG. 4 showing a distal end of the elongated probe having a larger diameter than in FIG. 3.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for using an elongated ultrasonic probe in conjunction with a radiopaque coating in order to improve the visibility of the ultrasonic probe during a procedure such as fluoroscopy. The radiopaque coating may be an ink comprising a substance which allows for a significant amount of x-ray absorption. The present invention provides a method of improving the visibility of an ultrasonic device during a fluoroscopic procedure comprising applying a radiopaque coating to an elongated probe having a small diameter. The radiopaque coating of the present invention is capable of withstanding vibrations of the elongated probe and increases the visibility of the elongated probe in a fluoroscopic procedure.

The following terms and definitions are used herein:

“Ablate” as used herein refers to removing, clearing, or destroying debris. “Ablation” as used herein refers to the removal, clearance, destruction, or taking away of debris.

“Cavitation” as used herein refers to shock waves produced by ultrasonic vibration, wherein the vibration creates a plurality of microscopic bubbles which rapidly collapse, resulting in a molecular collision by water molecules which collide with force thereby producing the shock waves.

“Non-radiopaque” as used herein refers to a material that does allow the passage of x-rays or other radiation.

“Radiopaque” as used herein refers to a material that does not allow the passage of x-rays or other radiation.

“Node” as used herein refers to a region of minimum energy emitted by an ultrasonic probe at or proximal to a specific location along the longitudinal axis of the probe.

“Anti-node” as used herein refers to a region of maximum energy emitted by an ultrasonic probe at or proximal to a specific location along the longitudinal axis of the probe.

“Probe” as used herein refers to a device capable of being adapted to an ultrasonic generator means, which is capable of propagating the energy emitted by the ultrasonic generator means along its length, resolving this energy into effective cavitational energy at a specific resonance (defined by a plurality of nodes and a plurality of anti-nodes at pre-determined locations along an “active area” of the probe) and is capable of acoustic impedance transformation of ultrasound energy to mechanical energy.

“Ultrasonic probe” as used herein refers to any medical device utilizing ultrasonic energy with the ability to ablate debris including, but not limited to, probes, elongated wires, and similar devices known to those skilled in the art. The ultrasonic energy of the ultrasonic probe may be in either a longitudinal mode or a transverse mode.

“Transverse” as used herein refers to vibration of a probe at right angles to the axis of a probe. A “transverse wave” as used herein is a wave propagated along an ultrasonic probe in which the direction of the disturbance at each point of the medium is perpendicular to the wave vector.

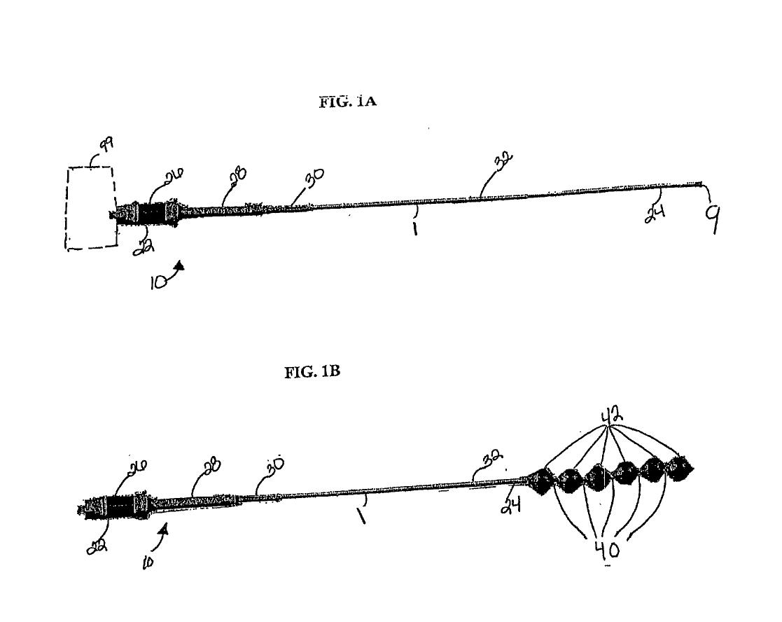

An ultrasonic medical device operating in a transverse mode of the present invention is illustrated generally at 10 in FIG. 1A. The ultrasonic medical device operating in a transverse mode includes an elongated probe 1 which is coupled to a device providing a source or a generator 99 (shown in phantom in FIG. 1A) for the production of ultrasonic energy. The ultrasonic generator 99 may or may not be a physical part of the ultrasonic medical device of the present invention itself A transducer 22 transmits ultrasonic energy received from the generator 99 to the probe 1. The probe 1 includes a proximal end 30 and a distal end 24. The transducer 22 is capable of engaging the ultrasonic probe 1 at the proximal end 30 with sufficient restraint to form an acoustical mass that can propagate the ultrasonic energy provided by the ultrasonic generator 99. The distal end 24 of the probe 1 is a thin terminal interval ending in a probe tip 9, which has a small diameter enabling the distal end 24 to flex longitudinally. The probe tip 9 can be any shape including, but not limited to, bent so that the probe tip 9 is not just longitudinal, or bigger shapes for removing a larger area of tissue. In one embodiment of the present invention shown in FIG. 1A, a diameter of the probe 1 decreases at defined intervals 26, 28, 30, and 32. Energy from the ultrasonic generator 99 is transmitted along the length of the probe 1, causing the probe 1 and the probe tip 9 to vibrate.

The transverse mode of vibration of the ultrasonic probe according to the present invention differs from the axial (or longitudinal) mode of vibration disclosed in the prior art. Rather than vibrating in the axial direction, the probe vibrates in a direction transverse (perpendicular) to the axial direction. As a consequence of the transverse vibration of the probe 1, the tissue-destroying effects of the device are not limited to those regions of a tissue coming into contact with the probe tip 9. Rather, as the active portion of the probe 1 is positioned in proximity to a diseased area or lesion, the tissue is removed in all areas adjacent to the multiplicity of energetic anti-nodes that are produced along the entire length of the probe 1, typically in a region having a radius of up to about 6 mm around the probe 1.

Transversely vibrating ultrasonic probes for tissue ablation are described in the Assignee's co-pending patent applications (U.S. Ser. No. 09/776,015, U.S. Ser. No. 09/618,352 and U.S. Ser. No. 09/917,471) which further describe the design parameters for such a probe and its use in ultrasonic devices for tissue ablation and the entirety of these applications are hereby incorporated by reference.

As a consequence of the probe design, as the ultrasonic energy propagates along the length of the probe 1 and along the probe terminal interval 32, the ultrasonic energy manifests as a series of transverse vibrations, rather than longitudinal vibrations. As shown in FIG. 1B, a plurality of nodes 40 occur along the length of the probe 1 and at the probe tip 9 at repeating intervals. The nodes 40 are areas of minimum energy and minimum vibration. A plurality of anti-nodes 42, or areas of maximum energy and maximum vibration, also occur at repeating intervals along the probe 1 and at the probe tip 9. The number of nodes 40 and anti-nodes 42, and their spacing along the probe 1 depends on the frequency of the energy produced by the ultrasonic generator 99. The separation of the nodes 40 and the anti-nodes 42 is a function of the frequency, and can be affected by tuning the probe 1. In a properly tuned probe 1, the anti-nodes 42 will be found at a position exactly one-half of the distance between the nodes 40 located adjacent each side of the anti-node 42. This will occur approximately for all tunings. The tissue-destroying effects of the ultrasonic medical device operating in a transverse mode of the present invention 10 are not limited to those regions of a tissue coming into contact with the probe tip 9. Rather, as the probe 1 is swept through an area of the tissue, preferably in a windshield-wiper fashion, the tissue is removed in all areas adjacent to the plurality of anti-nodes 42 being produced along the entire length of the probe 1. The extent of the cavitation energy produced by the probe tip 9 is such that it extends radially outward from the probe tip 9 at the anti-nodes 42 for about 1-6 millimeters. In this way, actual treatment time using the transverse mode ultrasonic medical device according to the present invention 10 is greatly reduced as compared to methods disclosed in the prior art.

By eliminating the axial motion of the probe and allowing transverse vibrations only, the active probe can cause fragmentation of large areas of tissue spanning the entire length of the active portion of the probe due to generation of multiple cavitational anti-nodes along the probe length perpendicular to the axis of the probe. Since substantially larger affected areas can be denuded of the diseased tissue in a short time, actual treatment time using the transverse mode ultrasonic medical device according to the present invention is greatly reduced as compared to methods using prior art probes that primarily utilize longitudinal vibration (along the axis of the probe) for tissue ablation. A distinguishing feature of the present invention is the ability to utilize probes of extremely small diameter (about 0.025 inches and smaller) compared to prior art probes, without loss of efficiency because the tissue fragmentation process in not dependent on the area of the probe tip (distal end). Highly flexible probes can therefore be designed to mimic device shapes that enable facile insertion into tissue spaces or extremely narrow interstices. Another advantage provided by the present invention is the ability to rapidly remove tissue from large areas within cylindrical or tubular surfaces.

A significant advantage of the present invention is that it physically destroys and removes adipose or other high water content tissue through the mechanism of non-thermal cavitation. The removal of tissue by cavitation also provides the ability to remove large volumes of tissue with a small diameter probe, without making large holes in the tissue or the surrounding areas. Accordingly, because of the use of cavitation as the mechanism for destroying tissue, together with the use of irrigation and aspiration, the method and apparatus of the present invention can destroy and remove tissue within a range of temperatures of ±7° C. from normal body temperature. Therefore, complications attendant with the use of thermal destruction or necrosis of tissue, such as swelling or edema, as well as loss of elasticity are avoided. Furthermore, the use of fluid irrigation can enhance the cavitation effect on surrounding tissue, thus speeding tissue removal.

The cavitation energy is the energy that is expelled from the probe in a stream of bubbles which must contact the tissue to cause ablation. Therefore, blocking the cavitation bubble stream from contacting tissue will spare the tissue from ablation, while directing the cavitation bubble stream to contact the tissue will cause ablation.

The number of nodes 40 and anti-nodes 42 occurring along the axial length of the probe is modulated by changing the frequency of energy supplied by the ultrasonic generator 99. The exact frequency, however, is not critical and the ultrasonic generator 99 run at, for example, 20 kHz is generally sufficient to create an effective number of tissue destroying anti-nodes 42 along the axial length of the probe. In addition, as will be appreciated by those skilled in the art, it is possible to adjust the dimensions of the probe 1, including diameter, length, and distance to the ultrasonic energy generator 99, in order to affect the number and spacing of the nodes 40 and anti-nodes 42 along the probe 1. The present invention allows the use of ultrasonic energy to be applied to tissue selectively, because the probe 1 conducts energy across a frequency range of from about 20 kHz through about 80 kHz. The amount of ultrasonic energy to be applied to a particular treatment site is a function of the amplitude and frequency of vibration of the probe 1. In general, the amplitude or throw rate of the energy is in the range of about 25 microns to about 250 microns, and the frequency in the range of about 20,000 Hertz to about 80,000 Hertz (20 kHz-80 kHz). In a preferred embodiment of the present invention, the frequency of ultrasonic energy is from about 20,000 Hertz to about 35,000 Hertz (20 kHz-35 kHz). Frequencies in this range are specifically destructive of hydrated (water-laden) tissues such as endothelial tissues, while substantially ineffective toward high-collagen connective tissue, or other fibrous tissues including, but not limited to, vascular tissues, epidermal, or muscle tissues.

In a preferred embodiment of the present invention, the ultrasonic generator 99 is mechanically coupled to the proximal end 22 of the probe 1 to oscillate the probe 1 in a direction transverse to its longitudinal axis. Alternatively, a magneto-strictive generator may be used for generation of ultrasonic energy. The preferred generator is a piezoelectric transducer that is mechanically coupled to the probe 1 to enable transfer of ultrasonic excitation energy and cause the probe 1 to oscillate in a transverse direction relative to its longitudinal axis. The ultrasonic medical device 10 is designed to have a small cross-sectional profile, which also allows the probe 1 to flex along its length, thereby allowing the probe 1 to be used in a minimally invasive manner. Transverse oscillation of the probe 1 generates a plurality of cavitation anti-nodes 42 along the longitudinal axis of the probe 1, thereby efficiently destroying the tissues that come into proximity with the energetic anti-nodes 42. A significant feature of the present invention resulting from the transversely generated energy is the retrograde movement of debris, e.g., away from the probe tip 9 and along the shaft of the probe 1.

The amount of cavitation energy to be applied to a particular site requiring treatment is a function of the amplitude and frequency of vibration of the probe 1, as well as the longitudinal length of the probe 1, the proximity of the probe 1 to a tissue, and the degree to which the probe 1 length is exposed to the tissue.

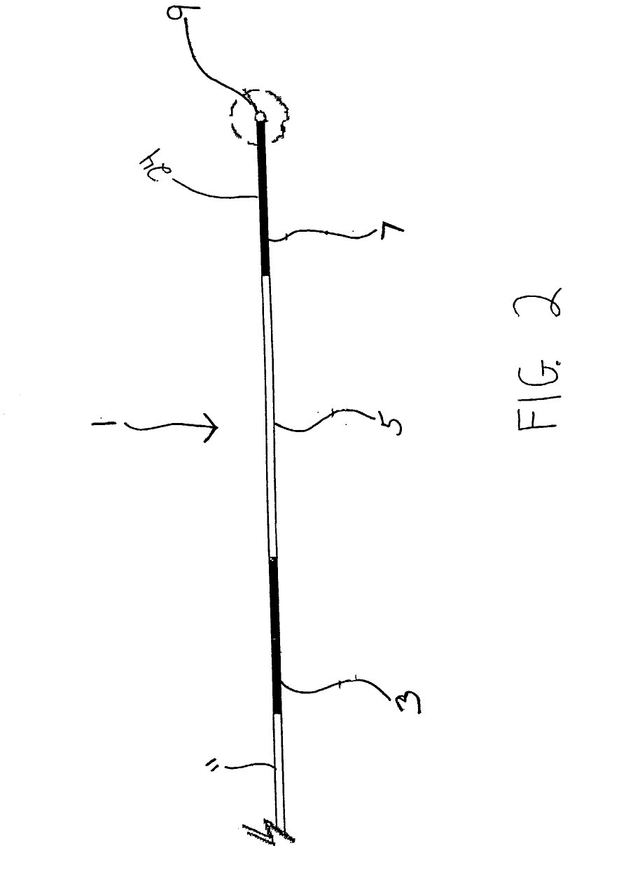

FIG. 2 shows an elongated probe 1 of the present invention with a radiopaque coating at a plurality of predetermined locations 3,7. The elongated probe of the present invention comprises a plurality of lengths 5,11 that are not coated with the radiopaque coating. The radiopaque coating is biocompatible and non-toxic. In a preferred embodiment of the present invention, the radiopaque coating is an ink. The radiopaque coating of the present invention is capable of withstanding vibrations of the elongated probe 1 and increases the visibility of the elongated probe 1 in a fluoroscopic procedure. The non-radiopaque, elongated probe 1 coated with a radiopaque coating allows the ultrasonic device 10 to continue to benefit from the high capacitance properties of a non-radiopaque probe and gain the ability to absorb radiation and therefore increase the visibility of the elongated probe 1 during a procedure such as fluoroscopy.

The radiopaque ink that may be used with the present invention is any ink that is high gloss, fast curing, and resistant to chemicals. The ink can be any pad printing ink including, but not limited to, Tampapur TPU or other similar acrylic based inks known in the art. In a preferred embodiment of the present invention, the radiopaque ink used to coat a plurality of predetermined locations 3,7 of the elongated probe 1 comprises Tampapur TPUL (commercially available from Marabuwerke GmbH & Co.; Tamm, Germany; www.marabu.com). In a preferred embodiment, Tampapur TPUL clear with tungsten powder additive is used as the radiopaque ink to coat the plurality of predetermined locations 3,7 of the elongated probe 1.

Tampapur TPUL is suited to print onto pre-treated polyethylene (PE) and polypropylene (PP), but also onto polyurethane (PU), polyamide (PA), melamine resins, phenolic resins, metal, anodized aluminum, coated substrates, powder-coated surfaces, wood, and glass. On polyacetal (POM), as for example Hostaform C or Derlin, a satisfying adhesion can be achieved by forced air drying (300-400° C., 3-4 sec.) Since all the print substrates mentioned may be different in printability, even within an individual type, preliminary trials are essential to determine the suitability for the intended use.

Tampapur TPUL is used when extremely high mechanical and chemical resistance on thermosetting plastics, polyethylene, polypropylene, and metals are required. When printing onto polyethylene and polypropylene, one must pretreat the surface of the substrate by flaming. One can achieve a very good adhesion with the Tampapur TPUL with a surface tension of at least about 42-48 mN/m.

Only pigments of high fade resistance are used in the Tampapur TPUL range. Shades mixed by adding overprint varnish or other color shades, and especially white, have a reduced fade and weather resistance depending on their mixing range. The fade resistance also decreases if the printed ink film thickness is reduced. The pigments used are resistant to solvents and plasticizers.

After proper and thorough drying, the ink film exhibits outstanding adhesion as well as rub, scratch and block resistance and is resistant to a large number of chemical products, oils, greases and solvents. Those skilled in the art will recognize that other inks could be used and still be within the scope of the present invention.

In an embodiment of the present invention, the radiopaque coating comprises tungsten (commercially available from Aldrich; www.sigmaaldrich.com) and epoxy (commercially available from Masterbond; Catalog No. EP3HTMED; www.masterbond.com) and the resulting mixture is used as the radiopaque ink to coat a plurality of predetermined locations 3,7 of the elongated probe 1. The tungsten used is monocrystalline, 0.6 to 1 μm (Aldrich Catalog No. 51, 010-6). In another embodiment of the present invention, radiopaque coating comprises a ratio of tungsten to epoxy at a ratio of 6:1. Those skilled in the art will recognize that other epoxy:tungsten ratios and other grades of tungsten and epoxy could be used and still be within the scope of the present invention.

In an embodiment of the present invention, the radiopaque ink comprises barium sulfate (commercially available from Sigma; Catalog No. B-8675; www.sigmaaldrich.com) and an epoxy to coat the plurality of predetermined locations 3,7 of the elongated probe 1. In another embodiment of the present invention, the radiopaque coating comprises gold. In another embodiment of the present invention, the radiopaque coating comprises tantalum. In another embodiment of the present invention, the radiopaque coating comprises an iodine-based compound. In another embodiment of the present invention, an x-ray absorbing compound is added to an epoxy base and the resulting mixture is used to coat the plurality of predetermined locations 3,7 of the elongated probe 1. Those skilled in the art will recognize that other similar materials or various grades of barium sulfate could be used as the radiopaque coating and still be within the scope of the present invention.

In another embodiment of present invention, an epoxy is used to coat the elongated probe 1 at a plurality of predetermined locations 3,7 wherein the epoxy is radiopaque. In an embodiment of the present invention, the epoxy comprises barium sulfate (commercially available from Masterbond, Catalog No. EP21MED; www.masterbond.com). Those skilled in the art will recognize that other similar materials could be used as in conjunction with epoxy and still be within the scope and spirit of the present invention.

The elongated probe 1 of the present invention is either a single diameter wire with a uniform cross section offering flexural stiffness along its entire length, or is tapered or stepped along its length to control the amplitude of the transverse wave along its entire longitudinal axis. Alternatively, the elongated probe 1 can be cross-sectionally non-cylindrical and capable of providing both flexural stiffness and support energy conversion along its entire length. The length of the elongated probe 1 of the present invention is chosen so as to be resonant in either in an exclusively transverse mode, or be resonant in combination of transverse and longitudinal modes. In a preferred embodiment of the present invention, the elongated probe 1 is chosen to be from about 30 centimeters to about 300 centimeters in length. In a most preferred embodiment of the present invention, the elongated probe 1 has a length of about 70 centimeters to about 210 centimeters in length.

As shown in FIG. 2, an elongated probe 1 of the present invention comprises a non-radiopaque material. Suitable probe 1 materials include metallic materials and metallic alloys suited for ultrasound energy transmission. In an embodiment of present invention, the elongated probe 1 is comprised of titanium. In another embodiment of the present invention, the elongated probe 1 is comprised of stainless-steel.

The elongated probe 1 of the present invention comprises a radiopaque coating at a plurality of predetermined location 3,7 along the length of the elongated probe 1. In one embodiment of the present invention, the elongated probe 1 comprises a radiopaque coating at a plurality of predetermined locations 3,7 wherein each predetermined location is approximately 0.200 inches in length and a first predetermined location is spaced approximately 0.850 inches away from a second predetermined location. In another embodiment of the present invention, the elongated probe 1 comprises a radiopaque coating at a plurality of predetermined locations 3,7 wherein each predetermined location 3,7 is approximately 0.500 inches in length and a first predetermined location 7 is spaced approximately 2.000 inches away from a second predetermined location 3. Those skilled in the art will recognize that many variations on the length of the radiopaque coated predetermined locations, the number of predetermined locations, and the spacing between the predetermined locations would be within the spirit and scope of the present invention. For example, the elongated probe 1 could have a number of predetermined locations from 1 to about 20 or more. The length of a predetermined location 3,7 could vary anywhere from about 0.001 inches up to about 2.000 inches or more.

In one embodiment of the present invention, a diameter of the predetermined locations 3,7 along the elongated probe 1 is approximately 0.012 inches (diameter of the elongated probe 1 in addition to the radiopaque coating.) In a preferred embodiment of the invention, the 0.012 diameter of the predetermined location 3,7 comprises a small amount of radiopaque coating so that the elongated probe 1 is used as a single-use medical device. In one embodiment, the 0.012 diameter of the predetermined location 3,7 comprises a sufficient amount of radiopaque coating so that the elongated probe 1 may be used multiple times. In one embodiment of the present invention, the combined diameter of the elongated probe 1 and the radiopaque coating is equal to or less than 0.025 inches. In another embodiment of the present invention, the diameter of the elongated probe 1 without a radiopaque coating varies from about 0.002 inches to about 0.025 inches. Those skilled in the art will appreciate that variations in diameter would be within the spirit and scope of the present invention. Visibility of the elongated probe 1 during a procedure such as fluoroscopy is improved as the amount of radiopaque coating is increased. Thus, a single radiopaque coating or multiple radiopaque coatings at each predetermined location are within the scope of the present invention. With each subsequent radiopaque coating, the overall diameter of the ultrasonic medical device 10 with multiple radiopaque coatings will increase. However, it is beneficial to limit the thickness of a radiopaque coating(s) and maintain a small-diameter profile of the elongated probe 1 to enable facile insertion of the probe into highly occluded or extremely narrow interstices within a blood vessel.

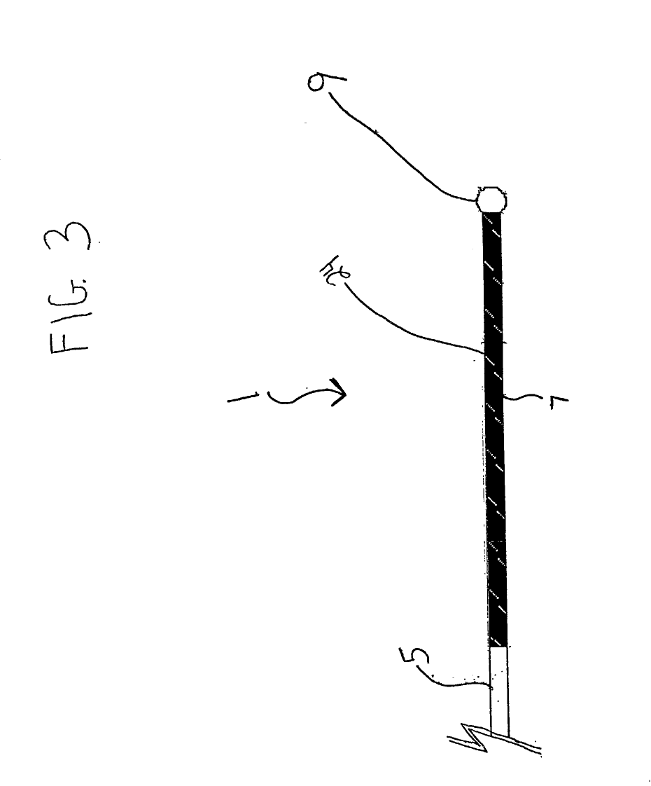

FIG. 3 shows an enlarged fragmentary view of the distal end 24 of the elongated probe 1 of FIG. 2. In a preferred embodiment of the present invention, the elongated probe 1 culminates in a probe tip 9. In one embodiment, the diameter of the probe tip 9 is approximately 0.016 inches. In a preferred embodiment, the probe tip 9 is not coated with a radiopaque coating. In one embodiment, the probe tip 9 is coated with a radiopaque coating. The probe tip 9 may be any shape including, but not limited to, a ball, a square, or a bent wire. Those skilled in the art will recognize that variations in the shape of the probe tip 9 would be within the scope and spirit of the present invention.

In a preferred embodiment of the present invention, the distal end 24 of the elongated probe 1 comprises a radiopaque coating a predetermined location 7 directly preceding the probe tip 9 wherein the probe tip 9 terminates the elongated probe 1. In an embodiment of the present invention, the predetermined location 7 directly preceding the probe tip 9 is approximately 0.200 inches in length. In one embodiment, the predetermined location 7 directly preceding the probe tip 9 is approximately 0.500 inches in length. Those skilled in the art will recognize that variations in the length of the predetermined locations would be within the scope and spirit of the present invention.

In an embodiment of the present invention, the elongated probe 1 culminates with a final predetermined location 7, coated with a radiopaque coating, leading into the probe tip 9. In another embodiment of the invention, the elongated probe 1 culminates with a length of the elongated probe 1 that has not been coated with a radiopaque coating.

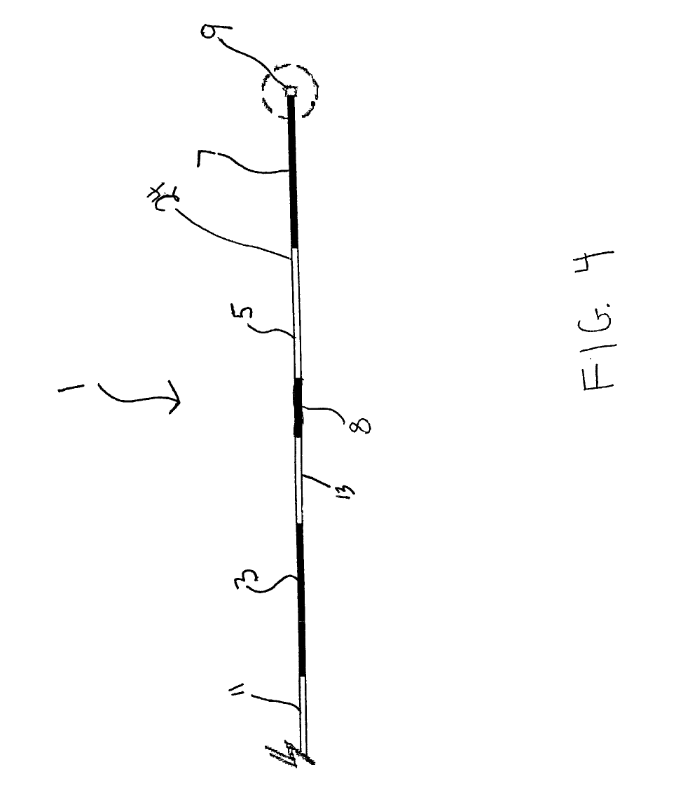

FIG. 4 shows an alternative embodiment of the elongated probe 1 of the present invention showing a large-diameter probe having three predetermined locations 3,7,8 coated with a radiopaque coating. The elongated probe of the present invention has a plurality of lengths 7,11,13 that do not comprise the radiopaque coating. In an embodiment of the invention, the elongated probe 1 comprises a plurality of predetermined locations 3,7,8 that have been coated with a radiopaque coating. In another embodiment of the present invention, each of the predetermined locations 3,7,8 has a different length. The multiple predetermined locations 3,7,8 can all have the same length or each can have a different length. The number of predetermined locations 3,7,8 can vary from 1 to about 20 or more. In a preferred embodiment of the present invention, the radiopaque coating is an ink. The elongated probe 1 with the radiopaque coating of the present invention is capable of withstanding vibrations of the elongated probe 1 and increases the visibility of the elongated probe 1 in a fluoroscopic procedure. The improved visibility in a fluoroscopic procedure of the elongated probe 1 coated with a radiopaque coating allows the ultrasonic device 10 to benefit from the high capacitance properties of a non-radiopaque probe 1 and gain the ability to absorb radiation with the aid of the radiopaque coating and therefore increase visibility in such procedures.

As shown in FIG. 4, the elongated probe 1 of the present invention comprises a radiopaque coating at a plurality of predetermined location 3,7,8 along the length of the elongated probe 1. In one embodiment of the present invention, the elongated probe 1 comprises a radiopaque coating at a plurality of predetermined locations 3,7,8 wherein each predetermined location 3,7,8 is approximately 0.200 inches in length and a first predetermined location is spaced approximately 7.0 inches away from a second predetermined location. Those skilled in the art will recognize that many variations on the length of the radiopaque coating, the number of predetermined locations, and the spacing between the predetermined locations would be within the spirit and scope of the present invention.

In an embodiment of the present invention, the diameter of the predetermined locations 3,7,8 along the elongated probe is approximately 0.018 inches (diameter of the elongated probe 1 in addition to the radiopaque coating.) In another embodiment of the present invention, the diameter of the elongated probe 1 without a radiopaque coating varies from about 0.010 inches to about 0.025 inches. In another embodiment of the present invention, the 0.018 diameter of the predetermined location 3,7,8 comprises a small amount of radiopaque coating so that the elongated probe 1 is used as a one time use medical device. In another embodiment of the present invention, the 0.018 diameter of the predetermined location 3,7,8 comprises a sufficient amount of radiopaque coating so that the elongated probe 1 may be used repeatedly. Those skilled in the art will recognize that variations in diameter would be within the spirit and scope of the present invention.

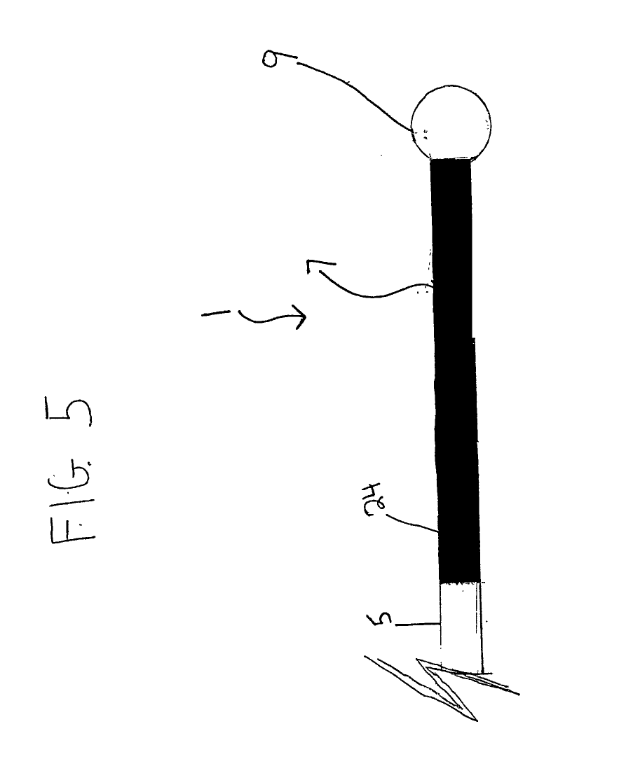

FIG. 5 shows an enlarged fragmentary view of an embodiment of the distal end 24 of the elongated probe 1 of FIG. 4 having a larger diameter. In an embodiment of the present invention, the elongated probe 1 culminates in a probe tip 9. In a preferred embodiment of the present invention, the probe tip 9 is not coated with a radiopaque coating. In another embodiment of the present invention, the probe tip 9 is coated with a radiopaque coating.

In a preferred embodiment of the present invention, the distal end 24 of the elongated probe 1 comprises a radiopaque coating coating a predetermined location 7 directly preceding the probe tip 9 wherein the probe tip 9 terminates the elongated probe 1. In another embodiment of the present invention, the predetermined location 7 directly preceding the probe tip 9 is approximately 0.200 inches in length. In another embodiment, the predetermined location 7 directly preceding the probe tip 9 is approximately 0.500 inches in length. Those skilled in the art will recognize that many variations on the length of the predetermined locations with a radiopaque coating, the number of predetermined locations, and the spacing between the predetermined locations would be within the spirit and scope of the present invention.

The present invention also includes a method of applying a radiopaque coating to an ultrasonic medical device. The benefits of the method of the present invention include, but are not limited to, providing a radiopaque coating on an elongated probe 1 wherein the radiopaque coating is capable of withstanding vibrations of the elongated probe 1 and increases the visibility of the elongated probe 1 in a fluoroscopic procedure. The improved visibility in a fluoroscopic procedure of the elongated probe 1 coated with a radiopaque coating allows the ultrasonic device 10 to benefit from the high capacitance properties of a non-radiopaque probe and gain the ability to absorb radiation with the aid of the radiopaque coating and therefore increase visibility in such procedures.

In a preferred method of the present invention, a radiopaque coating is applied to an elongated probe 1 at a plurality of predetermined location by a process of pad printing. The process of pad printing is probably the most versatile of all printing processes due to its unique ability to print on three-dimensional objects and compound angles. The theory behind the pad printing was derived from the screen, rubber stamp and photogravure printing process.

The first step in the process of pad printing is known as flooding. In the flooding step, the image to be transferred is etched into a primary plate commonly referred to as a cliche. Once mounted in the machine, the cliche is flooded with ink. The surface of the cliche is then doctored clean, leaving ink only in the image area. As solvents evaporate from the image area the ink's ability to adhere to the silicone transfer pad increases.

The second step in the process of pad printing is known as the pick up step. In the pick up step, the pad is positioned directly over the cliche, pressed on to it to pick up the ink, and then lifted away. The physical changes that take place in the ink during flooding (and wiping) account for the ink's ability to leave the recessed engraving in favor of the pad.

The third step is known as the print stroke step. After the pad has lifted away from the cliche to its complete vertical height, there is a delay before the ink is deposited on the substrate. During this stage, the ink has just enough adhesion to stick to the pad (it can easily be wiped off, yet it does not drip.) The ink on the pad surface once again undergoes physical changes: solvents evaporate from the outer ink layer that is exposed to the atmosphere, making it tackier and more viscous.

The fourth step in the process of pad printing is the ink deposit step. In the ink deposit step, the pad is pressed down onto the substrate, conforming to its shape and depositing the ink in the desired location. Even though it compresses considerably during this step, the contoured pad is designed to roll away from the substrate surface rather than press against it flatly. A properly designed pad, in fact, will never form a 0-degree contact angle with the substrate; such a situation would trap air between the pad and the part, resulting in an incomplete transfer.

The fifth and final step of the pad printing process is known as the pad release step. In this final step, the pad lifts away from the substrate and assumes its original shape again, leaving all of the ink on the substrate. The ink undergoes physical changes during the head stroke and loses its affinity for the pad. When the pad is pressed onto the substrate, the adhesion between the ink and the substrate is greater than the adhesion between the ink and the pad, resulting in a virtually complete deposit of the ink. This leaves the pad clean and ready for the next print cycle.

In a preferred method of the present invention, a radiopaque coating is applied to an elongated probe 1 at a plurality of predetermined location by a process of pad printing. In a preferred embodiment of the present invention, a pad printing procedure using the above-mentioned five steps is followed. In another embodiment of the present invention, one or more or the above-mentioned five steps may be omitted from the pad printing process. Those skilled in the art will recognize that additional steps may be added to the pad printing process and still be within the spirit and scope of the present invention.

In an embodiment of the present invention, a radiopaque coating is applied to an elongated probe 1 by inserting a non-radiopaque probe into a preshaped mold comprising the radiopaque coating. The first step of the molding process comprises injecting the radiopaque coating into the preshaped mold. The preshaped mold will provide for the desired number and length of predetermined locations along the elongated probe 1 to be coated with the radiopaque coating. The diameter of the preshaped mold may be varied to correspond to the desired diameter of the elongated probe 1 with the radiopaque coating. The second step of the molding process comprises inserting the elongated probe 1 into the preshaped mold. The third step of the molding process comprises allowing the elongated probe 1 to cure with the radiopaque coating. The elongated probe is cured at about 300° F. for approximately ten minutes. The fourth and final step comprises removing the elongated probe 1 comprising a plurality of predetermined locations with the radiopaque coatings from the preshaped mold.

In a method of the present invention, a radiopaque coating is applied to an elongated probe 1 at a plurality of predetermined location by the above-identified molding process. In a preferred embodiment of the present invention, a molding procedure using the above-mentioned steps is followed. In another embodiment of the present invention, one or more of the above-mentioned steps may be omitted from the above-identified molding process. Those skilled in the art will recognize that additional steps may be added to the molding process and still be within the spirit and scope of the present invention.

In an embodiment of the present invention, the elongated probe 1 undergoes preparation before the addition of the radiopaque coating. The preparation of the elongated probe 1 cleans the elongated probe 1, removes surface contamination, prevents corrosion, and forms a passive (less reactive) surface of the elongated probe 1. In an embodiment of the present invention, the elongated probe 1 undergoes a passivation before the addition of the radiopaque coating. In another embodiment of the present invention, the elongated probe undergoes an acid etch technique before the radiopaque coating is applied. Those skilled in the art will recognize that other preparation procedures known in the art would be within the spirit and scope of the present invention.

In an embodiment of the present invention, a radiopaque coating is applied to an elongated probe 1 at a plurality of predetermined locations 3,7,8 by a process of silk screening. In silk screening, a pattern is applied to a screen and an ink is transferred through a plurality of gaps in the screen onto the surface to be coated.

In an embodiment of the present invention, a radiopaque coating is applied to an elongated probe at a plurality of predetermined locations 3,7,8 by direct application of the radiopaque coating to the elongated probe 1. The radiopaque coating is applied to the elongated probe 1 by a number of methods including, but not limited to, painting the radiopaque coating onto the elongated probe 1, brushing the radiopaque coating onto the elongated probe 1, or dipping the elongated probe 1 into the radiopaque coating. Those skilled in the art will recognize that variations in the methods of applying the radiopaque coating to the elongated probe 1 are within the spirit and scope of the present invention.

The apparatus and the method of the present invention are useful in procedures including, but not limited to, barium x-rays, cardiac catheterization, and placement of intravenous (IV) catheters (hollow tubes into veins or arteries). In barium x-rays, fluoroscopy allows the physician to see the movement of the intestines as the barium moves through them. In cardiac catheterization, fluoroscopy enables the physician to see the flow of blood through the coronary arteries in order to evaluate the presence of arterial blockages. For intravenous catheter insertion, fluoroscopy assists the physician in guiding the catheter into a specific location inside the body. The present invention may also diagnose problems with the digestive tract, the bowel, kidneys, gallbladder, stomach, upper GI and joints. The apparatus and method of the present invention will facilitate a physician's ability to observe the action of an instrument being used either to diagnose or to treat a patient.

All references, patents, patent applications and patent publications cited herein are hereby incorporated by reference in their entireties. Variations, modifications, and other implementations of what is described herein will occur to those of ordinary skill in the art without departing from the spirit and scope of the present invention as claimed. Accordingly, the present invention is to be defined not by the preceding illustrative description but instead by the spirit and scope of the following claims.

What is claimed is:

1. An ultrasonic device comprising:
an elongated probe having a small-diameter; and
a radio-opaque coating wherein the radiopaque coating coats the elongated probe at at least one predetermined location and the radiopaque coating is capable of withstanding a series of vibrations of the elongated probe.

2. The device of claim 1 wherein the elongated probe comprises a plurality of predetermined locations on the elongated probe wherein the plurality of predetermined locations comprise the radiopaque coating.

3. The device of claim 2 wherein each of the plurality of predetermined locations on the elongated probe comprise a distinct radiopaque coating.

4. The device of claim 1 wherein the elongated probe comprises a plurality of predetermined locations having the radiopaque coating wherein each of the plurality of predetermined locations is spaced apart from each other by a length.

5. The device of claim 4 wherein the length between each of the plurality of predetermined locations are approximately equal.

6. The device of claim 4 wherein the length between each of the plurality of predetermined locations are not equal.

7. The device of claim 4 wherein each of the plurality of predetermined locations are approximately equal in length.

8. The device of claim 4 wherein each of the plurality of predetermined locations are not equal in length.

9. The device of claim 1 wherein the small-diameter of the elongated probe is small enough to be inserted into the vasculature of the body.

10. The device of claim 1 wherein the radiopaque coating is an ink.

11. The device of claim 10 wherein the ink comprises a mixture of Tampapur TPU and a tungsten powder.

12. The device of claim 10 wherein the ink comprises an adhesive material.

13. The device of claim 12 wherein the adhesive material is a biocompatible epoxy.

14. The device of claim 12 wherein the adhesive material comprises a substance that allows for a significant amount of x-ray absorption.

15. The device of claim 1 wherein the elongated probe comprises titanium.

16. The device of claim 1 wherein the elongated probe comprises stainless-steel.

17. The device of claim 1 wherein the elongated probe comprises a non-radiopaque material.

18. The device of claim 1 wherein the radiopaque coating is a nontoxic coating.

19. The device of claim 1 wherein the radiopaque coating is a biocompatible coating.

20. The device of claim 1 wherein the radiopaque coating comprises a material selected from the group consisting of gold, tantalum, tungsten, and barium sulfate.

21. The device of claim 1 wherein the radiopaque coating comprises an iodine-based compound.

22. The device of claim 1 wherein the radiopaque coating comprises tungsten.

23. The device of claim 1 wherein the radiopaque coating comprises Tampapur TPU.

24. An ultrasonic device comprising:
an elongated probe having a small-diameter and composed primarily of a non-radiopaque material; and
a radiopaque ink coating the elongated probe wherein the radiopaque ink is capable of withstanding vibrations of the elongated probe.

25. The device of claim 24 wherein the elongated probe comprises a plurality of predetermined locations having the radiopaque coating wherein each of the plurality of locations is approximately 0.5 inches in length and spaced approximately 2.0 inches apart from each other along a length of the elongated probe.

26. The device of claim 25 wherein the small-diameter of the plurality of predetermined location coated with the radiopaque ink is equal to or less than approximately 0.025 inches.

27. The device of claim 24 wherein the radiopaque ink comprises a mixture of Tampapur TPU and a tungsten powder.

28. The device of claim 24 wherein the radiopaque ink comprises an adhesive material.

29. The device of claim 28 wherein the adhesive material is a biocompatible epoxy.

30. The device of claim 28 wherein the adhesive material comprises a substance that allows for a significant amount of x-ray absorption.

31. The device of claim 24 wherein the radiopaque ink is a non-toxic ink.

32. The device of claim 24 wherein the radiopaque ink is a biocompatible ink.

33. The device of claim 24 wherein the radiopaque ink comprises a material selected from the group consisting of gold, tantalum, tungsten, and barium sulfate.

34. The device of claim 24 wherein the radio-opaque ink comprises an iodine-based compound.

35. The device of claim 24 wherein the elongated probe comprises a plurality of predetermined locations on the elongated probe wherein the plurality of predetermined locations comprise the radiopaque coating.

36. The device of claim 24 wherein the radiopaque ink comprises tungsten.

37. The device of claim 24 wherein the radiopaque ink comprises Tampapur TPU.

38. A method of improving the visibility of an ultrasonic device during a fluoroscopic procedure comprising:
applying a radiopaque coating to an elongated probe having a small-diameter wherein the radiopaque coating is an ink applied as a plurality of predetermined locations on the elongated probe.

39. The method of claim 38 wherein the plurality of predetermined locations are spaced apart from each other by a length.

40. The method of claim 39 wherein the length between each of the plurality of predetermined locations is approximately equal.

41. The method of claim 39 wherein the length between each of the plurality of predetermined locations is not equal.

42. The method of claim 39 wherein each of the plurality of predetermined locations are approximately equal in length.

43. The method of claim 39 each of the plurality of predetermined locations is not equal in length.

44. The method of claim 38 wherein the diameter of the elongated probe is small enough to be inserted into the vasculature of the body.

45. The method of claim 38 wherein the radiopaque coating is applied to the elongated probe by a process of pad printing.

46. The method of claim 38 wherein the radiopaque coating is applied to the elongated probe by a molding processes comprising placing an amount of the radiopaque coating into a preshaped mold, inserting the elongated probe into the preshaped mold, and removing the elongated probe with the plurality of predetermined locations having the radiopaque coating from the preshaped mold.

47. The method of claim 46 further comprising curing the elongated probe while the elongated probe is inserted in the preshaped mold.

48. The method of claim 38 wherein the radiopaque coating is applied to the elongated, probe by a process of silk screening.

49. The method of claim 38 wherein the radiopaque coating is applied to the elongated probe by a process of direct application.

50. The method of claim 38 wherein the ink comprises a mixture of Tampapur TPU and a tungsten powder.

51. The method of claim 38 wherein the ink comprises an adhesive material.

52. The method of claim 51 wherein the adhesive material is a biocompatible epoxy.

53. The method of claim 51 wherein the adhesive material comprises a substance that allows for a significant amount of x-ray absorption.

54. The method of claim 38 wherein the elongated probe comprises a plurality of predetermined locations having the radiopaque coating wherein each of the plurality of locations is approximately 0.5 inches in length and spaced approximately 2.0 inches apart from each other along a length of the elongated probe.

55. The method of claim 38 wherein the radiopaque coating comprises a biocompatible material.

56. The method of claim 38 wherein the elongated probe comprises titanium.

57. The method of claim 38 wherein the elongated probe comprises stainless-steel.

58. The method of claim 38 wherein the radiopaque coating is a nontoxic material.

59. The method of claim 38 wherein the radiopaque coating comprises a material selected from the group consisting of gold, tantalum, tungsten, and barium sulfate.

60. The method of claim 38 wherein the radiopaque coating comprises an iodine-based compound.

61. The method of claim 38 further comprising applying multiple layers of the radiopaque coating to the elongated probe.

62. The method of claim 38 wherein the elongated probe comprises a plurality of predetermined locations on the elongated probe wherein the plurality of predetermined locations each comprise the radiopaque coating.

63. The method of claim 62 wherein each of the plurality of predetermined locations on the elongated probe comprise a distinct radiopaque coating.

64. The method of claim 38 further comprising applying a single use radiopaque coating to the elongated probe and disposing of the elongated probe after a single use.

65. The method of claim 38 wherein the ink comprises tungsten.

66. The method of claim 38 wherein the ink comprises Tampapur TPU.

67. A method-of improving the visibility of an non-radiopaque, elongated probe when the elongated probe is inserted in a body comprising applying a radiopaque coating to the elongated probe at a plurality of predetermined locations along the elongated probe.

68. The method of claim 67 wherein the radiopaque coating is applied to the elongated probe by a process of pad printing.

69. The method of claim 67 wherein the radiopaque coating is applied to the elongated probe by a molding processes comprising placing an amount of the radiopaque coating into a preshaped mold, inserting the elongated probe into the preshaped mold, and removing the elongated probe with the plurality of predetermined locations having the radiopaque coating from the preshaped mold.

70. The method of claim 69 further comprising curing the elongated probe while the elongated probe is inserted in the preshaped mold.

71. The method of claim 67 wherein the radiopaque coating is an ink.

72. The method of claim 71 wherein the ink comprises a biocompatible epoxy.

73. The method of claim 71 wherein the ink comprises a mixture of Tampapur TPU and a tungsten powder.

74. The method of claim 71 wherein the ink comprises a material that allows for a significant amount of x-ray absorption.

75. The method of claim 67 further comprising applying multiple layers of the radiopaque coating to the plurality of predetermined locations.

76. The method of claim 67 wherein the radiopaque coating comprises a material selected from the group consisting of gold, tantalum, tungsten, and barium sulfate.

77. The method of claim 67 wherein the radiopaque coating comprises an iodine-based compound.

78. The method of claim 67 wherein the diameter of the elongated probe coated with the radiopaque coating is equal to or less than approximately 0.025 inches in diameter.

79. The method of claim 67 wherein the radiopaque coating comprises tungsten.

80. The method of claim 67 wherein the radiopaque coating comprises Tampapur TPU.
